(19) United States
(12) Patent Application Publication
(10) Pub. No.: US 2023/0307134 A1
(43) Pub. Date: Sep. 28, 2023

(54) SYSTEM AND METHOD FOR AUTOMATED ASSESSMENT OF EMOTIONAL STATE OF AN EMPLOYEE USING ARTIFICIAL INTELLIGENCE

(71) Applicant: CULTURE SOFTWARE SOLUTIONS, INC., NEWPORT COAST, CA (US)

(72) Inventor: JEFFREY S. MARKS, Newport Coast, CA (US)

(21) Appl. No.: 18/189,721

(22) Filed: Mar. 24, 2023

Related U.S. Application Data
(60) Provisional application No. 63/324,390, filed on Mar. 28, 2022.

(51) Int. Cl. G16H 50/20

(52) U.S. Cl. CPC G16H 50/20

(57) ABSTRACT

A method and a system for automated assessment of emotional state of an employee using artificial intelligence, is disclosed. The method includes receiving a login request by an employee on an automated assessment platform and providing a actionable prompt to employee via a user interface and receiving an action from the employee on the actionable prompt and capturing employee assessment data based on the actions received and analyzing the captured employee assessment data by the AI engine to assess an emotional state of the employee using one or more artificial intelligence models. The method includes determining by the AI engine remedial action for the employee, based on analysis, to facilitate maintaining of emotional state of the employee. The AI engine selects a remedial content to be displayed to the employee or determines an action to be taken by a manager and intimates the manager to perform the action.

### CROSS-REFERENCE TO RELATED APPLICATIONS

This application claims priority to U.S. provisional patent application No. 63/324,390, filed on 28 Mar. 2022, the complete disclosure of which, in its entirely, is herein incorporated by reference.

### BACKGROUND

### Technical Field

The embodiments herein generally relate to human resource management systems. More particularly, the embodiments herein relate to an automated system and method for automated assessment of emotional state of an employee using artificial intelligence.

### Description of the Related Art

Typically, human resource workers, managers, or supervisors, serve as a liaison between employers and employees, for example, in many circumstances a human resource personnel may have to deal with the employees when they fail to work efficiently or are unhappy, so as to prevent them from quitting or continuing to perform inefficiently. Many factors influence employee sentiment, consequently affecting performance or work culture. Often the human resource personnel are unable to accurately assess or address the lack of interest or poor performance of an employee. Therefore, there is a need for automated solutions for assessing the issues associated with the employees and provide motivation to the employees to improve work culture and improve efficiency of the employees. Moreover, employers have limited visibility into employee sentiment and emotional state. COVID-19 has compelled companies to re-imagine workplace environments and respond to shifting employee expectations. Those expectations are evolving to include more emphasis on the mental health aspects of employee wellness programs. Despite efforts to adapt, a gap is growing between employee expectations about work environments and organizational priorities, and current methods many organizations use to measure and address employee sentiment do not adequately address this gap. Without a consistent understanding of what employees want, it is difficult to meet their expectations.

Notably, frequent monitoring of employee sentiment can enable employers to address problems associated with employees in real-time and help employees feel valued, heard and appreciated by management. Companies that are more attuned to emotional health and sentiment of their employees are better able to attract, engage and retain employees.

Currently, there are several employee engagement software platforms in the market that gather employee feedback through electronic surveys, and which include a dashboard where management can review, sort, and analyze data gathered through electronic surveys. Most of the surveys are anonymous and, therefore, cannot be matched to specific employees. Also, most of these platforms deploy surveys on a monthly, quarterly, or annual basis, and therefore, are not gathering real time data about the mood and sentiment of employees. Further, none of the existing solutions include a customizable trigger that provides real time notifications to designated persons if an individual employee's sentiment or mood is low, and which therefore provides employers with real-time information enabling them to intervene when an employee's sentiment or mood is low. Additionally, existing techniques use infrequent data collection, look backward, are not actionable, are hard to implement and provide limited solutions to assist companies in taking actions to improve employee sentiment and overall company culture.

Hence there is a need for a method and a system to automatically assess emotional well-being of employees on a regular basis, that enables identifying potential problem areas while also facilitating targeted actions that can be taken by the company based on real-time data.

The above-mentioned shortcomings, disadvantages and problems are addressed herein, and which will be understood by reading and studying the following specification.

### SUMMARY

This summary is provided to introduce a selection of concepts in a simplified form that are further disclosed in the detailed description. This summary is not intended to determine the scope of the claimed subject matter.

In one aspect, a processor-implemented method of automated assessment of emotional state of an employee using artificial intelligence is provided. The method includes receiving a login request by an employee on an automated assessment platform. The method also includes generating a actionable prompt via an artificial intelligence engine (AI engine), associated with a server, may be based on one or more attributes associated with the employee and providing the actionable prompt to the employee via a user interface by the automated assessment platform upon login. The method also includes capturing employee assessment data, by the automated assessment platform, based on one or more actions of the employee in response to the actionable prompt and transmitting the employee assessment data to the AI engine. The method also includes analyzing the employee assessment data, by the AI engine, to assess the emotional state of the employee using one or more artificial intelligence models. The method also includes determining a remedial action for the employee, by the AI engine, based on analysis, to facilitate maintaining of emotional state of the employee.

In an embodiment herein, generating the actionable prompt includes analyzing a previously gathered data associated with the employee and one or more attributes associated with the employee by the AI engine and generating the actionable prompt in real-time for each individual employee based on the analysis.

In an embodiment herein, the actionable prompt is generated based on geofencing.

In an embodiment herein, the actionable prompt is personalized based on one or more attributes associated with the employee.

In an embodiment herein, the method further includes progressively learning by the AI engine about the employee based on the employee assessment data collected over a period of time and predict one or more attributes associated with the employee based on the learning.

In an embodiment herein, the method further includes calculating an employee performance and/or sentiment score by the AI engine, based on one or more data points associated with the captured actions.

In an embodiment herein, the method further includes determining the personalized remedial content suitable for the employee for improving the emotional state or performance of the employee.

In an embodiment herein, the method further includes identifying by the AI engine one or more patterns in the employee assessment data that is predictive of pone or more attributes associated with an employee using the employee assessment data, using one or more machine learning techniques.

In an embodiment herein, the method further includes generating one or more alert/notification signals by the automated assessment platform, upon the employee performance and/or sentiment score being below a predetermined threshold.

In an embodiment herein, capturing employee assessment data includes capturing at least one of an identification data comprising at least one of: a facial recognition data, a voice recognition data, a fingerprint data, of the employee upon the employee logging into the automated assessment platform and transmitting the identification data to the AI engine.

In an embodiment herein, the method further includes determining by the AI engine an emotional state of the employee based on the facial recognition data, voice data, question responses, or any or all of the foregoing, and triggering a notification based on the emotional state determined.

In an embodiment herein, the method further includes capturing a narrative response to one or more questions, from the employee upon the employee logging into the automated assessment platform, transmitting the narrative to the AI engine, and determining by the AI engine an emotional state of the employee based on the narrative data and triggering a notification based on the emotional state determined.

In an embodiment herein, the method further includes weighing, by the AI engine, one or more individual assessment scores corresponding to at least one of: mood, physical energy, and/or emotions, of the employee, based on one or more statistical techniques and models.

In an embodiment herein, determining the remedial action includes selecting remedial content to be displayed to at least one of the employee and the manager.

In an embodiment herein, determining the remedial action includes determining an action to be taken by a manager and intimating the manager by the automated assessment platform to perform the action.

In an embodiment herein, the actionable prompt comprises audio and/or video content displayed to the employee, wherein a response from the employee in the form of at least one of: a) questions, b) video, c) audio or d) facial expression of the employee in response to the question, audio and/or video displayed to them, is captured by the automated assessment platform 106 for assessment.

In an embodiment herein, the actionable prompt is personalized in realtime by the AI engine based on one or more attributes associated with the employee.

In an embodiment herein, the AI engine is configured to analyze an aggregate employee performance or emotional state score, either alone, or together with certain previously aggregated employee performance or emotional state scores for at least one of: same or similar groups of employees and generate the actionable prompts in realtime for each individual employee based on the analysis.

In an embodiment herein, the AI engine is configured to analyze an aggregate employee performance or emotional state score, either alone, or together with certain previously aggregated employee performance or emotional state scores for a groups of employees and generate at least one of: a remedial content and one or more actions to be taken by a supervisor or management, for improving the emotional state of the group of employees.

In another aspect, a method of training an artificial intelligence engine for automated assessment of emotional state or performance of an employee using artificial intelligence is provided. The method includes providing an actionable prompt to an employee via a user interface by an automated assessment platform upon login by the employee on the automated assessment platform. The method also includes receiving one or more actions from the employee in response to the actionable prompt. The method also includes capturing employee assessment data based on one or more actions of the employee on the actionable prompt. The method also includes progressively training one or more machine learning models associated with an artificial intelligence engine using the captured employee assessment data, for assessing the emotional state or performance of the employee using the one or more artificial intelligence models. The one or more machine learning models are trained progressively based on employee assessment data captured in a plurality of instances over a predetermined period of time to identify one or more patterns that are predictive of performance of the employees.

In yet another aspect, a system for automated assessment of emotional state or performance of an employee using artificial intelligence is disclosed. The system includes an automated assessment platform comprising a processor and a memory comprising one or more executable modules executable by the processor for enabling automated assessment of emotional state of an employee using artificial intelligence. The automated assessment platform is configured for a) receiving a login request by an employee on an automated assessment platform; b) rendering an actionable prompt to the employee via a user interface; and c) capturing employee assessment data, based on one or more actions of the employee on the actionable prompt and transmitting the employee assessment data to an artificial intelligence (AI) engine associated with a server. The system also includes the server communicatively coupled to the automated assessment platform and comprising the AI engine and a database. The AI engine is configured to a) generate the actionable prompt in real-time based on one or more attributes associated with the employee, upon login by the employee on the automated assessment platform and b) receive employee assessment data, based on one or more actions of the employee on the actionable prompt, and c) analyze the employee assessment data, to assess the emotional state of the employee using one or more artificial intelligence models and d) generate a remedial content to be rendered to the employee based on analysis, to facilitate maintaining of emotional state of the employee.

In an embodiment herein, the AI engine is further configured to analyze previously gathered data associated with the employee by the AI engine and generate the actionable prompt in real-time for each individual employee based on the analysis.

In an embodiment herein, the AI engine is further configured to analyze previously gathered data associated with the employee together with one or more attributes associated with the employee by the AI engine and generate the actionable prompt in real-time for each individual employee based on the analysis.

In an embodiment herein, the AI engine is further configured to progressively learn about the employee based on the employee performance or emotional state data collected over a period of time and predict employee performance.

In an embodiment herein, the AI engine is further configured to calculate an employee performance or emotional state score based on one or more data points associated with the captured actions and determine the personalized remedial content to deliver to the employee, suitable for the employee for improving the emotional state of the employee.

In an embodiment herein, the AI engine is further configured to calculate an employee performance or emotional state score based on one or more data points associated with the captured actions and determine the personalized remedial content and actions to be taken by a supervisor or management, and which content is delivered to the supervisor or management, and which is suitable for the employee for improving the emotional state of the employee.

In an embodiment herein, the AI engine is further configured to identify one or more patterns in the employee assessment data that is predictive of performance of an employee using the employee assessment data, using one or more machine learning techniques.

In an embodiment herein, the automated assessment platform is further configured to generate one or more alert/notification signals by the automated assessment platform, upon the employee performance or emotional state score being below a predetermined threshold.

In an embodiment herein, the automated assessment platform is further configured to: capture a facial recognition data of the employee upon the employee logging into the automated assessment platform and transmit the facial recognition data, either alone, or along with the employee assessment data to the AI engine.

In an embodiment herein, the AI engine is further configured to determined, an emotional state of the employee based on the facial recognition data and triggering a notification based on the emotional state determined.

In an embodiment herein, the AI engine is further configured to determine an emotional state of the employee based on the voice data and triggering a notification based on the emotional state determined.

In an embodiment herein, the AI engine is further configured to determine an emotional state of the employee based on employee narrative response to one or more questions and triggering a notification based on the emotional state determined.

In an embodiment herein, the AI engine is further configured to determine an emotional state of the employee based on two or more of (a) employee narrative response to one or more questions, (b) facial recognition data, and (c) employee narrative response to one or more questions, and triggering a notification based on the emotional state determined.

In an embodiment herein, the AI engine is further configured to weigh individual scores for mood, physical energy, and/or emotions based on one or more statistical techniques and models.

In an embodiment herein, the AI engine is further configured to select a remedial content to be displayed to the employee.

In an embodiment herein, the AI engine is further configured to determine an action to be taken by a manager and intimate the manager via the automated assessment platform to perform the action and intimate the manager via the automated assessment platform to perform the action.

In an embodiment herein, the above methods can be aggregated and calculate an aggregate employee performance or emotional state score of a group of employees based on the data points associated with the captured inputs from the group of employees and determines a personalized remedial content for the group of employees based on the aggregate employee performance score.

In an embodiment herein, the AI engine is further configured to analyze such aggregate employee performance or emotional state score, either alone, or together with certain previously aggregated employee performance or emotional state scores for the same or similar groups of employees, and generate the actionable prompts in realtime for each individual employee based on the analysis.

In an embodiment herein, the AI engine is further configured to analyze such aggregate employee performance or emotional state score, either alone, or together with certain previously aggregated employee performance or emotional state scores for the same or similar groups of employees, and generate remedial content and actions to be taken by a supervisor or management, and which content is delivered to the supervisor or management, and which is suitable for the group of employees for improving the emotional state of such group of employees.

It is to be understood that the aspects and embodiments of the disclosure described above may be used in any combination with each other. Several of the aspects and embodiment herein may be combined to form a further embodiment herein of the disclosure.

The various embodiments of the present technology provides a unique mood, energy and emotion monitoring system as an independent web-based app that also integrates with point-of-sale, scheduling, time tracking systems and other computing systems used by employees.

The present technology captures a daily pulse of employee sentiment that provides a single source of truth, delivering actionable insights into employee emotional state. Using the present technology, the managers can quickly identify potential problem areas and take targeted actions based on real-time data. Additionally, the dashboards provide data tools, training and prescriptive solutions driven by machine learning algorithms. The present technology enables the managers to set alerts that are triggered based on threshold scores of inputs by the employees.

The foregoing summary is illustrative only and is not intended to be in any way limiting. In addition to the illustrative aspects, embodiments herein, and features described above, further aspects, embodiments herein, and features will become apparent by reference to the drawings and the following detailed description.

These and other objects and advantages will become more apparent when reference is made to the following description and accompanying drawings.

### BRIEF DESCRIPTION OF THE DRAWINGS

The other objects, features and advantages will occur to those skilled in the art from the following description of the preferred embodiment herein and the accompanying drawings in which:

FIG. 1 depicts a block diagram for a system for automated assessment of emotional state of an employee using artificial intelligence, in accordance with an embodiment herein;

FIG. 2 is a block diagram depicting the automated assessment platform accessed by one or more employees via their individual user devices, in accordance with an exemplary scenario in an embodiment herein;

FIG. 3 depicts a block flow diagram depicting a process of automated assessment of emotional state of an employee using artificial intelligence, in accordance with an embodiment herein;

FIG. 4 depicts a block flow diagram depicting the process of improving performance in an enterprise using the system of the present technology, in accordance with an embodiment herein:

FIG. 5 depicts an exemplary user interface view displayed to the employee upon logging into the automated assessment platform, in accordance with an exemplary scenario in an embodiment herein;

FIG. 6 depicts an exemplary view of a dashboard of an employee, in accordance with an exemplary scenario in an embodiment herein;

FIG. 7 depicts an example of a notification that would be sent to a manager by the automated assessment platform 106 upon the employee selecting a value in the user interface view, in accordance with an exemplary scenario in an embodiment herein;

FIG. 8 depicts a flowchart of a method of training one or more machine learning models for automated assessment of emotional state of an employee using artificial intelligence, in accordance with an embodiment herein;

FIG. 9 depicts a flowchart of a method automated assessment of emotional state of an employee using artificial intelligence, in accordance with an embodiment herein; and

FIG. 10 depicts a representative hardware environment for practicing the embodiments herein.

Although the specific features of the embodiments herein are shown in some drawings and not in others. This is done for convenience only as each feature may be combined with any or all of the other features in accordance with the embodiments herein.

### DETAILED DESCRIPTION OF THE DRAWINGS

The detailed description of various exemplary embodiments of the disclosure is described herein with reference to the accompanying drawings. It should be noted that the embodiments herein are described herein in such details as to clearly communicate the disclosure. However, the number of details provided herein is not intended to limit the anticipated variations of embodiments herein; on the contrary, the intention is to cover all modifications, equivalents, and alternatives falling within the spirit and scope of the present disclosure as defined by the appended claims.

It is also to be understood that various arrangements may be devised that, although not explicitly described or shown herein, embody the principles of the present disclosure. Moreover, all statements herein reciting principles, aspects, and embodiments of the present disclosure, as well as specific examples, are intended to encompass equivalents thereof.

While the disclosure is susceptible to various modifications and alternative forms, specific embodiment herein thereof has been shown by way of example in the drawings and will be described in detail below. It should be understood, however that it is not intended to limit the disclosure to the forms disclosed, but on the contrary, the disclosure is to cover all modifications, equivalents, and alternatives failing within the scope of the disclosure.

Various embodiments herein provide a system and a method for automated assessment of emotional state of an employee using artificial intelligence. The present technology provides an automated mood, energy and emotion monitoring system as an independent web-based app that also integrates with point-of-sale, scheduling and time tracking systems. The present technology captures pulse of employee sentiment at frequent intervals, such as during login and log off into the work environment, that provides a single source of truth, delivering actionable insights into employee state. Using the present technology, the managers can quickly identify potential problem areas and take targeted actions based on real-time data. Additionally, the dashboards provide data tools, training and prescriptive solutions driven by machine learning algorithms. Moreover, the present technology enables the managers to set alerts that are triggered based on threshold scores of inputs by the employees.

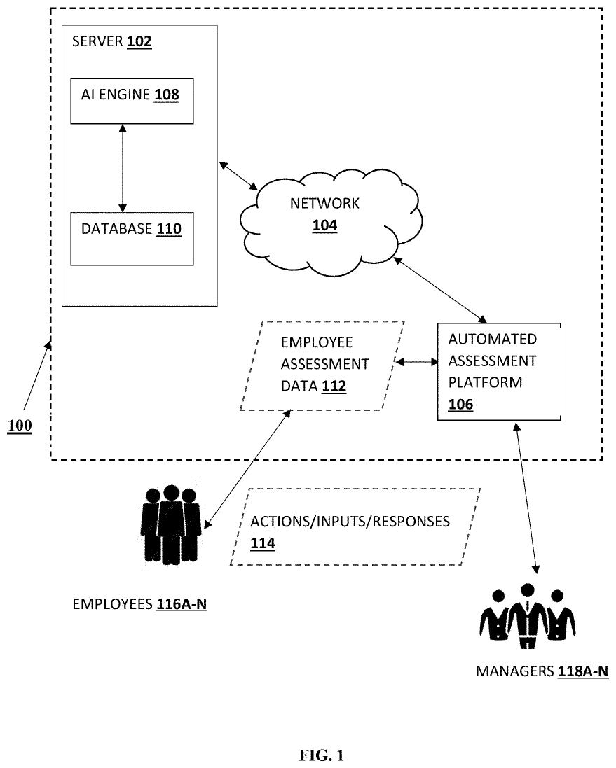

FIG. 1 depicts a block diagram for a system 100 for automated assessment of emotional state of an employee using artificial intelligence, in accordance with an embodiment herein. The term “employee” can be used interchangeably with the term “user” throughout the specification and may refer to any user of the system 100. The system 100 comprises a server 102 communicatively associated via a network 104, with an automated assessment platform 106. The server 102 includes an artificial intelligence (AI) engine 108 and a database 110. The network 104 may be for example, a private network and a public network, a wired network or a wireless network. The wired network may include, for example Digital Subscriber Line (DSL), coaxial cable lines, or optical fiber lines. The wireless network may include for example Bluetooth®, Bluetooth Low Energy (BLE), ANT/ANT+, ZigBee, Z-Wave, Thread, Wi-Fi®, Worldwide Interoperability for Microwave Access (WiMAX®), mobile WiMAX®, WiMAX®-Advanced, a satellite band and other similar wireless networks. The wireless networks may also include any cellular network standards to communicate among mobile devices. The database 110 comprises a repository of at least one of data associated with the employees/any person associated with an enterprise (such as managers), video files, audio files, text to motivate the employees and the like. In an embodiment herein, the database 110 may include content associated with for example, a chief executive officer (CEO) of the company as well, that can be further customized using a various types of software so that the employees can be displayed customized/personalized content from the CEO, or mail from CEO to motivate the employees. In some embodiments herein, the data associated with the employees/any person associated with an enterprise is meta-tagged in the repository. In some embodiments herein, the repository may be accessible directly by the employees or the data in the repository may be sent through the manager, so that the manager can be kept in loop in case of any trigger. In some embodiments herein, the automated assessment platform 106 may be installed on a client device accessed by a user/employee. In some other embodiments herein, the automated assessment platform 106 may be installed on a local server accessed by a user/employee through a client device. The automated assessment platform 106 can be implemented in the form or either software or hardware. In some embodiments herein, the automated assessment platform 106 is implemented as an application accessible via user devices of one or more employees 116A-N and/or one or more managers 118A-N of an enterprise. The term “employees” may also include interns of the enterprise, or any other person associated with any role in an enterprise.

In some embodiments herein, the system 100 may be employed outside of the work environment to, among other things, classrooms, social groups, business groups, religious organizations or other groups, whether formal or informal, where it may be beneficial to have real-time data on the sentiment or mood of the individuals within such environments or groups.

In an embodiment herein, upon signing up on the automated assessment platform 106, a company or organization is allowed to register an account at the automated assessment platform 106. The company admin can set up supervisors and managers 118A-N in the automated assessment platform 106 and assign them to a location. In an embodiment herein, the company/organization receives a unique QR code that can be displayed near point-of-sale systems or locations where the employees 116A-N check-in for their shifts.

In an embodiment herein, the employee logs into the automated assessment platform 106. In an embodiment herein, the employee may log into the automated assessment platform 10 by scanning a QR code, by accessing a mobile application or a mobile device, point of sale (POS), text links, by accessing an email, and any other known techniques of rendering a platform that can be accessed through a user interface. The employees 116A-N may log into the automated assessment platform 106 at the time of clocking-in/clocking out of office. In some embodiments herein, the employees may receive an email or text or any other type of intimation, upon clocking-in or clocking-out. In some embodiments herein, the automated assessment platform 106 may be integrated with scheduling, time tracking or payroll systems for providing timely intimations to the employees 116A-N. Upon logging in, the automated assessment platform 106 displays a user interface view that prompts the employees to provide one or more indicators of their moods in the form of for example, response to specific questions, selections of inputs from (for example a list of emotions (e.g. happy, sad, calm, agitated, excited, angry, respected, frustrated, trusted, disengaged, hopeful, depressed, and the like), numerical indicators of mood (for example, on an 11-point scale), their physical energy (for example, on an 11-point scale). In some embodiments herein, the AI engine 108 customizes one or more changes in the user interface view or the actionable prompt displayed via the user interface view in real-time based on one or more attributes associated with the employee. For example, upon determining that an employee is fond of color yellow which makes him happy, the AI engine 108 changes the background color of the user interface view of the employee to yellow. In some embodiments herein, upon logging in, the automated assessment platform 106 prompts the employees to take a quick survey rating their mood and physical energy on a scale of numbers (e.g., 11-point scale (0-10)). In some embodiments herein, the automated assessment platform 106 presents the employees with a follow up question asking them to record any emotions they are experiencing at that moment. In addition, the employee may be provided with an open-text box to share their concerns, suggestions. The one or more employees 116A-N and/or managers 118A-N of an enterprise may log into the automated assessment platform 106 at any time during the working hours, for instance they may log in during the start of the day, while ending the day of work, or any time during work hours as well based on their needs.

In some embodiments herein, the automated assessment platform 106 is configured to capture employee assessment data 112 from one or more employees 116 A-N based on actions, inputs or responses 114 received from one or more employees 116 A-N. In an embodiment herein, the automated assessment platform 106 provides an actionable prompt to the employee via a user interface displayed to the employee upon logging into the automated assessment platform 106. As used herein the term “actionable prompt” refers to a prompt rendered via the user interface to the requesting action from the employee in the form of for example, text input, selection of options, audio response, physical actions, and the like. In an embodiment herein, the actionable prompt is personalized based on the data associated with the employee in the database 110 and the employee assessment data 112. Examples of the actionable prompt may include, but is not limited to a question, a questionnaire, a capture of facial expressions, a prompt seeking action by the employee, a video displayed to the employee, a prompt asking the employee to select a value from a drop-down menu or a list of values indicative of mood of the employee, and the like. In some embodiments herein, the actionable prompt is personalized by the AI engine 108 for each employee accessing the automated assessment platform 106 using artificial intelligence and based on employee assessment data 112. In an embodiment herein, the actionable prompt is personalized and generated in real-time by the AI engine 106 upon the employee logging into the automated assessment platform 106 and transmitted to the automated assessment platform 106 to be rendered to the employee. In some embodiments herein, the actionable prompt is randomly generated by the AI engine 108 based on one or more attributes associated with the employee. The attributes may include for example, responses/actions/inputs received from the employee previously during a predetermined duration, for example, responses received over a period of one-week, emotional state of the employee, employee behavioral data, employee performance, past data associated with the employee, such as position, role in the company, length of employment, culture, prior data collected from that employee, and the like. In some embodiments herein, the actionable prompt is personalized in real-time by the AI engine based on one or more attributes associated with the employee.

In an embodiment herein, the actionable prompt includes audio and/or video rendered to the employee that requires the employees to respond back with a video or the audio and/or facial expression of the employee in response to the audio and/or video displayed to them and is captured by the automated assessment platform 106 for assessment. In some embodiments herein, the captured response is transmitted to the server 102. In an embodiment herein, the actionable prompt is generated based on geofencing. Geofencing is a location-based service that businesses use to engage their audience by sending relevant messages to smartphone users who enter a pre-defined location or geographic area. In an embodiment herein, the AI engine 108 generates a actionable prompt, when the employees trigger a search in a particular geographic location, enter a mall, neighborhood, or store, or when the employees enter a predefined geographical area. The AI engine 108 analyses the actions of the employee in response to the actionable prompt and generates a remedial content to be rendered to the employee based on analysis, to facilitate maintaining of emotional state of the employee. The remedial content may include, but is not limited to, audio, video, text, any combination thereof, and the like. In some embodiments herein, the remedial content may be obtained from the database 110. In some other embodiments herein, the remedial content may be gathered from one or more third party sources, such as for example a website displaying motivational content.

In some embodiments herein, an aggregate employee data is as an aggregate for a shift. The AI engine 108 analyses the aggregate data to provide suggestion to the manager on how to improve the overall energy of the employees corresponding to the shift.

In some embodiments herein, an aggregate employee data is as an aggregate for an entire department or location. The AI engine 108 analyses the aggregate data to provide suggestion to the manager on how to improve the overall mood and state of the employees corresponding to such department or location.

In some embodiments herein, an aggregate employee data is as an aggregate for an entire company. The AI engine 108 analyses the aggregate data to provide suggestion to management on how to improve the overall mood and state of all the employees within the company.

In some embodiments herein, the automated assessment platform 106 displays a questionnaire (a form of actionable prompt) to the employees 116 A-N upon login and captures the employee assessment data 112 from one or more employees 116 A-N based on the responses received for the questionnaire. In some embodiments herein, the questionnaire is personalized using artificial intelligence based on employee assessment data 112 (such as for example, monthly performance and other data associated with the employee, e.g., whether an employee is depressed or not motivated), follow up questions to know why the employee is going into a rabbit hole, and the like. The questions are selected from a repository of questions in the database 110. In an embodiment herein, an algorithm is employed to determine the questions that are relevant for a particular employee based on employee data in the repository/database 110.

In an embodiment herein, the algorithm determines one or more data points for a predetermined period of time (such as few years back) for each employee and so that they can set a baseline. In some embodiment herein, the automated assessment platform 106 may be configured to monitor the activities, such as the actions 114, performed by each of the employees 116 A-N on each of the associated user devices. In some cases, the automated assessment platform 106 may monitor the activities of the one or more employees 116 A-N by accessing an interface, such as an application programming interface (API) or the like, associated with the system 100. In some embodiments herein, the automated assessment platform 106 sends employee assessment data 112 to the server 102 at a predetermined interval, based on determining that a particular event has occurred, or any combination thereof. For example, the automated assessment platform 106 may send employee assessment data 112 to the server 102 at a predetermined interval, such as at hourly, weekly, or monthly intervals or the like.

In an embodiment herein, the capturing the employee assessment data 112, includes capturing at least one of an identification data comprising at least one of: a facial recognition data, a voice recognition data, a fingerprint data, of the employee upon the employee logging into the automated assessment platform and transmitting the identification data to the AI engine 108.

The server 102 receives the employee assessment data 112 and stores employee assessment data 112 in the database 110. In some embodiments herein, the AI engine 108 may include multiple AI engines. As used herein the term AI engine 108 refers to several fundamental modules which include a Machine Learning Module, a Natural Language Processing Module and a Knowledge Representation (Ontology) Module. The AI engine 108 may use one or more types of machine learning algorithms including, for example, supervised learning algorithms (e.g., classifier, regression, ensemble), unsupervised learning algorithms. K-nearest neighbors, random forest, artificial neural network, decision tree, support vector machine (SVM), reinforcement learning, linear regression, logistic regression, classification, and regression trees (CART), I Bayes, Bayesian network, and the like.

In some embodiments herein, each of the one or more employees 114AN may have a corresponding employee identifier. The server 102 may store the employee assessment data 112 with a previously gathered data and use the AI engine 108 to determine one or more attributes associated with each employee ID. For example, the previously gathered data may be cumulative and may include employee assessment data collected over a period of time, such as a large portion of the employee's career working at the enterprise. The AI engine 108 may analyze the previously gathered data and one or more attributes to determine a remedial content that can be generated and displayed for each individual employee.

In an embodiment herein, the questions displayed to the employees 116A-N on the user interface are customized using artificial intelligence by the AI engine 106, based on employee assessment data 112 (such as for example, monthly performance and other data associated with the employee (e.g., whether an employee is depressed or not motivated), follow up questions to know why the employee is going into a rabbit hole, and the like. In an embodiment herein, the AI engine 108 selects the questions from a repository of questions in the database 110. In an embodiment herein, an algorithm is employed to determine which questions are relevant for a particular employee based on employee data in the repository. In an embodiment herein, the algorithm determines one or more data points for a predetermined period of time (such as few years back) for each employee and so that they can set a baseline. In an embodiment herein, the automated assessment platform 108 may display a list of selectable icons or emojis to the employee that designate certain emotions such user is feeling. In an embodiment herein, the user interface may allow employees 116A-N to write a narrative and the AI engine 106 may determine the mental or emotional state of the employees 116A-N from the text. In an embodiment herein, a narrative response to one or more questions, is captured from the employee upon the employee logging into the automated assessment platform 106, the narrative is transmitted to the AI engine 108 and an emotional state of the employee is determined by the AI engine 108 based on the narrative data and triggering a notification based on the emotional state determined.

In some embodiments herein, the automated assessment platform 106 includes a dashboard to monitor based on the trigger points. For instance, if an employee is not well the manager is made aware through the dashboard and the manager may opt to change the content displayed to the employee that is unwell. In some embodiments herein, the manager may be tagging content through the directory. In an embodiment herein, the manager may meta tag the content and add his own meta tag as well, such that the remedial content generated from the database 110 (or central repository) or could be even through the manager using same content or changed content trough managers library of content, can be a subset of the database 110. The meta tagging can be manual or automated.

In some embodiments herein, the employee assessment data 112 is stored in the database 110 and is associated with a unique identifier (representing each employee). In some embodiments herein, the automated assessment platform 106 records comments, concerns, suggestions by the employees 116A-N in an open-text field. The automated assessment platform 106 passes on the employee assessment data 112 to the AI engine 108. The AI engine 108 calculates an employee performance score based on the data points associated with the captured inputs and determines a personalized remedial content for the employee that can help improve the sentiments or mood of the employee. In an embodiment herein, the AI engine 108 calculates an aggregate employee performance score of a group of employees based on the data points associated with the captured inputs from the group of employees and determines a personalized remedial content for the group of employees based on the aggregate employee performance score.

In an embodiment herein, upon the aggregate employee performance score being below a base line, the AI engine 108 may select a particular content to be shared to the employee or may send indicators for the managers to take collective action on the group of employees or share some content with the group of employees.

In an embodiment herein, the AI engine 108 is further configured to analyze such aggregate employee performance or emotional state score, either alone, or together with certain previously aggregated employee performance or emotional state scores for the same or similar groups of employees and generate the actionable prompts in real-time for each individual employee based on the analysis.

In an embodiment herein, the AI engine 108 is further configured to analyze aggregate employee performance or emotional state score, either alone, or together with certain previously aggregated employee performance or emotional state scores for the same or similar groups of employees, and generate remedial content and actions to be taken by a supervisor or management, and which content is delivered to the supervisor or management, and which is suitable for the group of employees for improving the emotional state of such group of employees.

In an embodiment herein, the AI engine 108, verifies if an employee or a group of employees are being truthful in the actions or responses to the actionable prompt by analyzing on one or more attribute associated with the responses or actions received from the employees, such as for example, a comparison one a current response with one or more previous responses of the employee as stored in the repository, a facial expression or body language of the employee and the like.

In some embodiments herein, the AI engine 108 progressively learns about the employee based on the employee performance data 112 collected over a period of time and predicts employee performance. The AI engine 108 analyses the employee performance data 112 using one or more trained AI models and generates personalized remedial content (such as motivational videos/audios) to be rendered to the employee for improving the mood/sentiment of the employee. The automated assessment platform 106 renders the personalized remedial content to the employee or directs the employee to personalized remedial content to help them manage their mood/emotions. The personalized remedial content may include, for example, mental health resource of the company, motivating videos, content that enhance emotional strength of the employee, and the like. In some embodiments herein, the employee may be prompted with a follow up calendar invite, inviting them to a meeting with their supervisor, human resource manager and the like based on the calculated employee performance score.

In some embodiments herein, the AI engine 108 progressively learns about an employee or a group of employees based on the employee performance data 112 collected over a period of time and determines a personality type of the employee or a group of employees.

In some embodiments herein, the AI engine 108 uses a machine learning based algorithm to adaptively learn how to adjust the trigger thresholds for individual employees, by comparing the employee assessment data 112 with some predefined data in the database 110. In some embodiment herein, the AI engine 108, adaptively learns to determine whether specific employees 116A-N are truthfully responding to surveys based on employee assessment data 112 collected over a period of time, for example over a week, a month, or few months. In some embodiments herein, the AI engine 108 adjusts individual trigger threshold for an employee, by analyzing data of other employees, in addition to the individual employee.

In some embodiments herein, the machine learning models associated with the AI engine 108 are trained to identify patterns that will be predictive of an employee's performance or emotional state using the employee assessment data 112. The performance score also triggers a recommendation engine that will generate one or more suggestions/recommendations for employees 116A-N and supervisors. For employees 116A-N, recommendations will include, but not be limited to, actions the employee can take to help improve performance (e.g., read an article, watch a video, issue a surprise/delight reward, and the like,) or emotional state; or actions the manager can take to help improve employee performance (e.g., talk to employee, give them a break (say for example, a 15 minutes break), schedule a one-on-one, and the like). The AI engine 108 records whether the employee took any action (e.g., read the article, watched the video) and whether the supervisor took any action (e.g., they recorded their interactions/interventions with the employee). In an embodiment herein, all the employee assessment data is fed back into the machine learning models to improve predictive capabilities.

In some embodiments herein, upon the employee performance score being below a predetermined threshold, the automated assessment platform 106 generates one or more alert/notification signals, for example, a low performance score alert is generated and sent to a supervisor and the supervisor may be directed to content that can help the supervisor address the concerns associated with the employee. In some embodiments herein, the automated assessment platform 106 generates participation reports, scores by location, supervisor/manager, date range, employee performance trend analysis, and the like and transmits to a senior manager. In an embodiment herein, an admin is allowed to select threshold levels below which managers/supervisors 118A-N will be sent a text alerts. In an embodiment herein, the automated assessment platform 106 provides a follow up question on whether a supervisor/manager has made a personal reach out to any employee after the assessment on two or more shifts after the alerts are triggered.

In some embodiments herein, the automated assessment platform 106 captures a facial recognition data of the employee upon the employee logging into the automated assessment platform 106. The automated assessment platform 106 transmits the facial recognition data along with the employee assessment data 112 to the AI engine 108. The AI engine 108 determines an emotional state of the employee based on the facial recognition data and triggers a notification based on the emotional state determined. The notification can be for example, that the employee is depressed, or is extremely happy, and the like. In some embodiments herein, the AI engine 108 uses the facial recognition data for determining the emotional state of the employee. In some embodiments herein, the automated assessment platform 106 captures facial recognition data over a period of time, such as few days and the AI engine 108 determines if there is any disparity between the response by the employee and the emotional state of the employee completing the questionnaire in connection with responses, to determine the emotional state of the employee and subsequently triggers a notification if there is disparity. In some embodiments herein, the AI engine 108 compares and correlates the facial recognition data to the responses/actions from the employees 116A-N to determine whether employees 116A-N are responding truthfully to the questions.

In some embodiments herein, the AI engine 108 may consider attributes such as (a) prior employee responses, (b) certain attributes of that employee, and/or (c) comparing individual data with other employee data, for determining determine whether employees 116A-N are responding truthfully to the questions. In an embodiment herein, the facial recognition data is captured from an image capture device external to the system 10) but communicatively associated with the automated assessment platform 106.

In an embodiment herein, the employee assessment data 112 comprises an aggregate employee data that is an aggregate for a shift, wherein the AI engine analyses the aggregate data to provide suggestion to the manager on how to improve the overall energy of the employees corresponding to the shift. In an embodiment herein, the aggregate employee data is as an aggregate for at least one of: an entire department, location, an entire company, and the like.

In some embodiments herein, the automated assessment platform 106 weighs individual performance scores of the employees 116A-N for assessing mood, physical energy, and/or emotions based on one or more statistical techniques and models. In an embodiment herein, the automated assessment platform 106 collects data from open-text fields that the employee uses to record their concerns, issues, suggestions and transmits the collected data to the AI engine 108. In some embodiments herein, the employee may provide text, voice, or audio in response to the actionable prompt. The AI engine 108 leverages natural language processing and other text-analytics or voice analytics to extract key emotional cues from the collected data associated with the employee. In some embodiments herein, one or more machine learning models are developed for the AI engine 108 to establish individual norms (for example, one employee's mood corresponding to a score of “8” may be different from another employee's mood corresponding to a score of “8”). These data in combination will create an emotional performance score for each employee.

In some embodiments herein, the automated assessment platform 106, displays questions to the employees 116A-N based on a geo-location of employee, for example when the employee walks into the work environment first time for the day. In an embodiment herein, the automated assessment platform 106 incorporates a dashboard that displays the current and historical responses of individuals or a group of individuals, and views comparing or ranking responses of different individuals or groups of individuals, over various periods of time. In an embodiment herein, the automated assessment platform 106 integrates into third-party electronic time-clock systems, point of sale systems, company computer networks, messaging applications, social media applications, electronic collaboration hubs or other digital platforms or similar systems. In an embodiment herein, the automated assessment platform 106 delivers questions to the employee, when the employee logs into, clocks into, or otherwise accesses a third-party platform. In an embodiment herein, the employee is prompted to complete the questionnaire as a condition to logging into, clocking into, or otherwise accessing a third-party platform. In an embodiment herein, the automated assessment platform 106, deploys the questions once each day, when an individual logs into, clocks into or otherwise accesses a third-party platform. In an embodiment herein, the automated assessment platform 106, may set a trigger notification if a response by the employee satisfies a specified threshold in one action or in a specified number of actions within specified period of time. In an embodiment herein, the automated assessment platform 106 may specify whether trigger notifications are delivered in real time, or at a specified time each day, each week, or each month. In an embodiment herein, the automated assessment platform 106 may send a separate notification for each individual whose response(s) satisfy the specified threshold, or one notification for all individuals whose response(s) satisfy the specified threshold, in each case during some specified period of time. In an embodiment herein, individual data including, among other things, individual names and/or identification numbers, and group affiliation, are automatically uploaded directly from a third-party platform into a secure database associated with or housed within the system 100. In an embodiment herein, the individual data, including, among other things, individual names and/or identification numbers, and group affiliation, may be manually uploaded in various electronic formats into a secure database associated with or housed within the system 100. In an embodiment herein, the individual data may be manually removed or added to the system 100 by an organization administrator through a user interface. In an embodiment herein, the thresholds or scores which trigger electronic notifications, may be set, or changed by the organization through a user interface. The system 100 may be implemented in a variety of computing systems, such as a mainframe computer, a server, a network server, a laptop computer, a desktop computer, a notebook, a workstation, and the like.

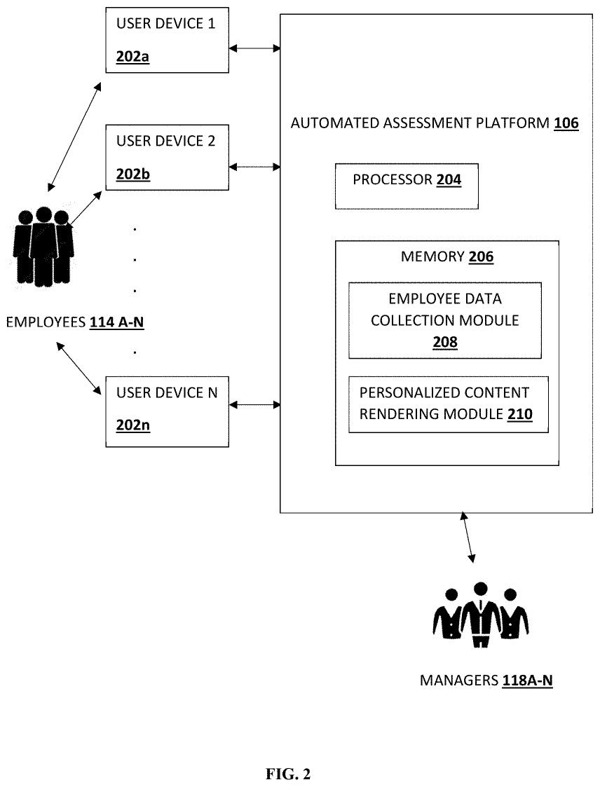

FIG. 2 is a block diagram depicting the automated assessment platform 106 accessed by one or more employees 116A-N via their individual user devices 202an, in accordance with an exemplary scenario. In an embodiment herein, the user devices 202a-n may include a software system, such as, for example, a customer relationship management (CRM) system, a help desk system, or another type of software system. Examples of the user devices 202a-n includes but is not limited to user devices (such as cellular phones, personal digital assistants (PDAs), handheld devices, laptop computers, personal computers, an Internet-of-Things (IoT) device, a smart phone, a machine type communication (MTC) device, a computing device, a drone, or any other portable or non-portable electronic device. The automated assessment platform 106 may include a processor 204 and a memory 206 storing one or more modules executable by the processor 204 for automated assessment of emotional state of an employee using artificial intelligence, in accordance with an embodiment herein. The one or more executable modules includes an employee data collection module 208 and a personalized content rendering module 210. The employee data collection module 208 is configured to capture employee assessment data 112 based on actions/inputs/responses 114 by the employee. The personalized content rendering module 210 is configured to render a personalized remedial content determined suitable for the employee by the AI engine 106 for improving the emotional state of the employee.

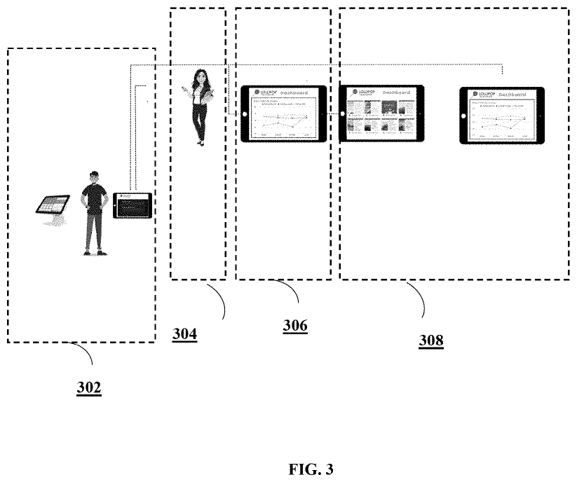

FIG. 3 depicts a block flow diagram depicting a process of automated assessment of emotional state of an employee using artificial intelligence, in accordance with an embodiment herein. At stage 302, a user (such as an employee) is prompted to complete a quick survey rating his/her mood. At stage 304, a low score in the response form the employee triggers alert to a manager and the manager checks in with the employee and taps into their dashboard to observe the trends and identify one or more practical solutions for the user. At stage 306, the manager analyzes user performance scores individually or in aggregate, compare average sentiment across staff shifts, and locations, and monitor changes in sentiment of the user. At stage 308, the manager receives a set of easy to implement and practical tools from the AI engine 106 to optimize performance of the users. The managers 118A-N are provided access to a library of leadership content targeted to front-line managers.

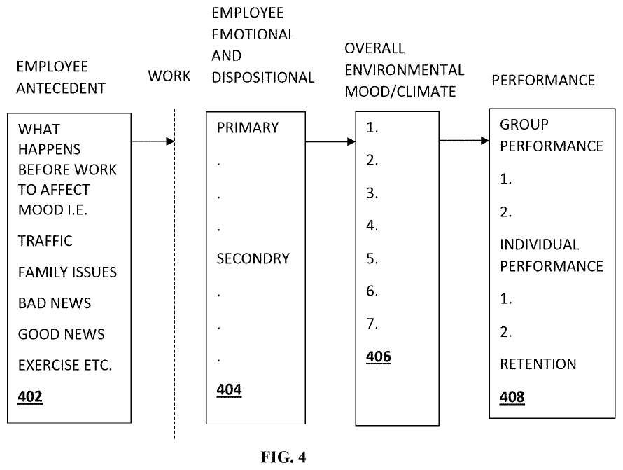

FIG. 4 depicts a block flow diagram depicting a process of improving performance in an enterprise using the system 100 of the present technology, in accordance with an embodiment herein. At stage 402, the AI engine 108 creates a performance score based on employee antecedents such as for example, what happens before work to affect mood, including for example traffic, family issues, bad news, good news, exercise, and the like. At stage 404, the automated assessment platform 106 assesses emotional disposition of the employee based on the performance score. At stage 406, the automated assessment platform 106 offers mood changers/enhancers to the employee. At stage 408, the automated assessment platform 106 enhances overall environmental mood climate for the employee on a scale of 1-7. At stage 410, the automated assessment platform 106 enables increasing performance by generating positive results and enhancement in for example, group performance, individual performance, retention, happiness, and the like.

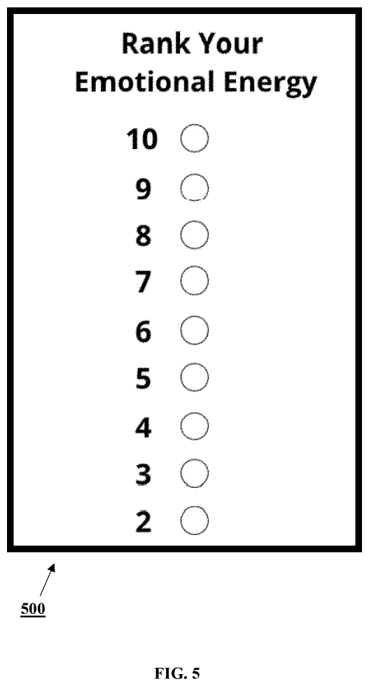

FIG. 5 depicts an exemplary user interface view 500 displayed to the employee upon logging into the automated assessment platform 106, in accordance with an exemplary scenario. As depicted in the user interface view 500, the employee is displayed a question asking him to rank his emotional energy/mood on a scale of 2-10.

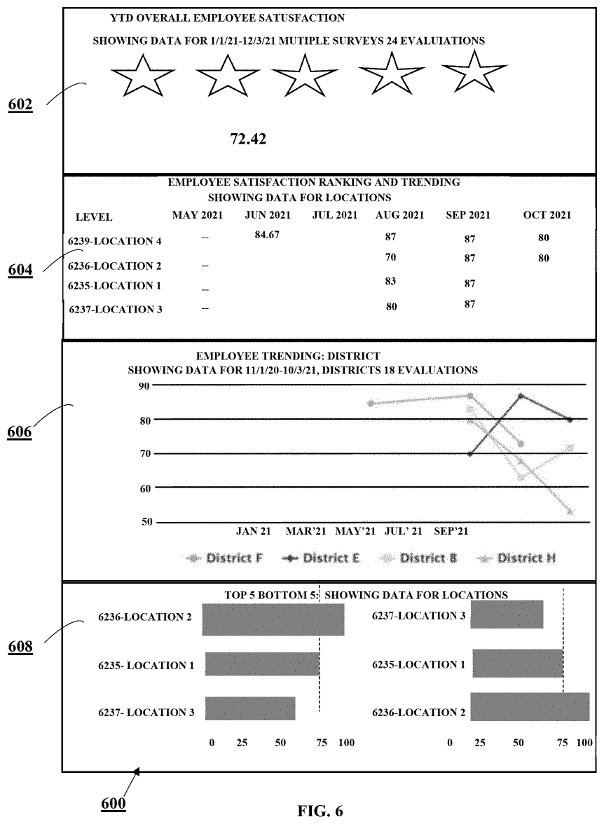

FIG. 6 depicts an exemplary view of a dashboard 600 of an employee, in accordance with an exemplary scenario. The exemplary view of the dashboard 600 includes, (a) an overall organization score over a specified period 602, (b) scores for each group over a specified period 604, (c) trending of scores over certain periods of time by one or more groups 608, and (d) top and bottom groups over a certain period of time 610.

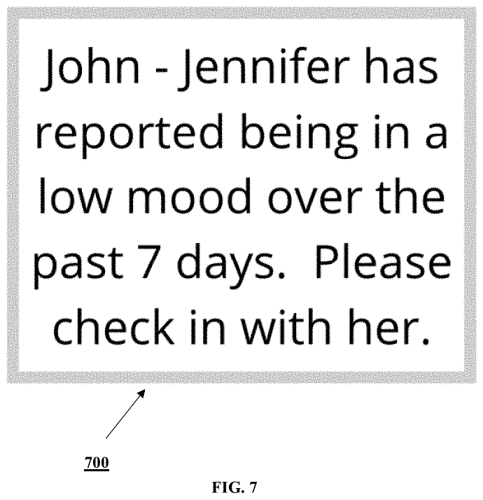

FIG. 7 depicts an example of a notification 700 that would be sent to a manager by the automated assessment platform 106 upon the employee selecting a value less than 5 in the user interface view 500, in accordance with an exemplary scenario.

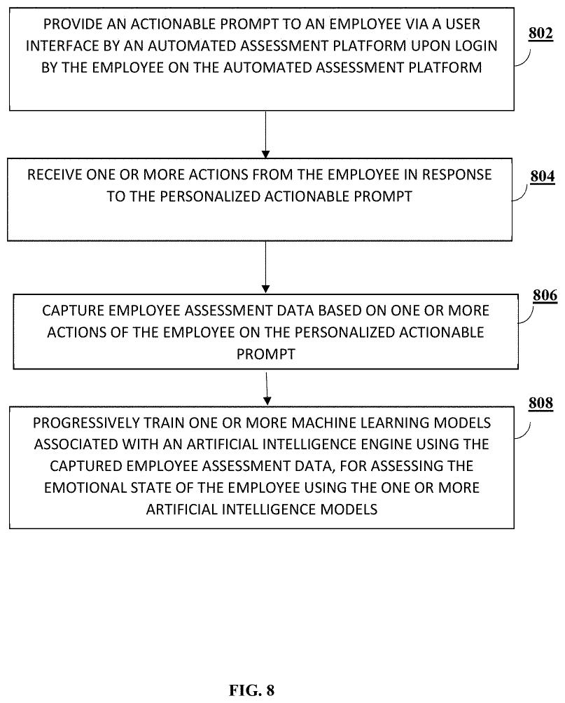

FIG. 8 depicts a flowchart of a method of training an AI model for automated assessment of emotional state of an employee using artificial intelligence, in accordance with an embodiment herein. At step 802, a actionable prompt is provided to an employee via a user interface by an automated assessment platform upon login by the employee on the automated assessment platform. At step 804, one or more actions received from the employee in response to the actionable prompt. At step 806, employee assessment data is captured based on one or more actions of the employee on the actionable prompt. At step 808, one or more machine learning models associated with an artificial intelligence engine are progressively trained using the captured employee assessment data, for assessing the emotional state of the employee using the one or more artificial intelligence models. The one or more machine learning models are trained progressively based on employee assessment data captured in a plurality of instances over a predetermined period of time to identify one or more patterns that are predictive of performance of the employees.

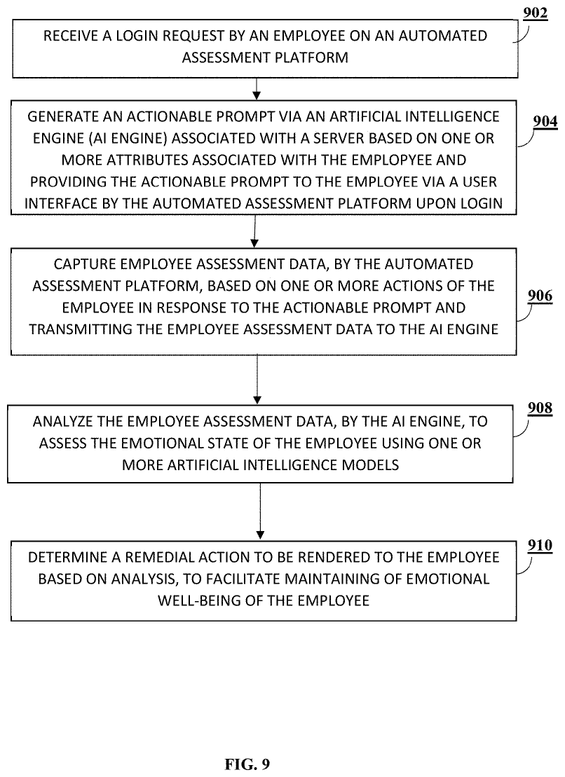

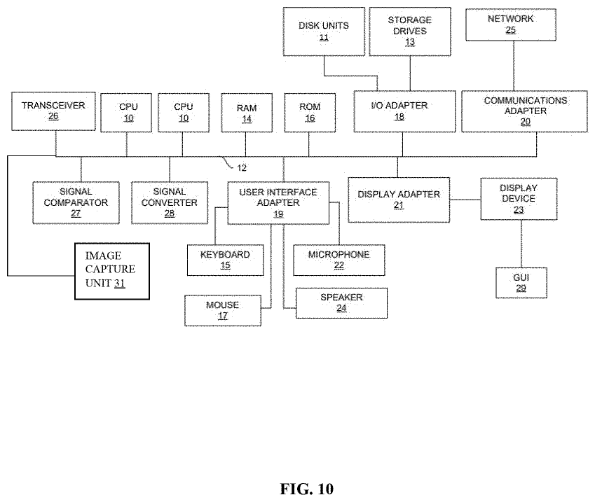

FIG. 9 depicts a flowchart of a method of automated assessment of emotional state of an employee using artificial intelligence, in accordance with an embodiment herein. At step 902, an automated assessment platform receives a login request by an employee. At step 904, the automated assessment platform provides a actionable prompt to the employee via a user interface. The actionable prompt may include, but is not limited to a question, a questionnaire, a capture of facial expressions, a prompt seeking action by the employee, a prompt asking the employee to select a value from a drop-down menu or a list of values indicating mood of the employee, and the like. At step 906, the automated assessment platform receives an action from the employee on the actionable prompt and captures employee assessment data based on the actions received. The actions may include for example response to a query, facial expression, physical actions, and the like. At step 908, the actions are analyzed by the AI engine to assess the emotional state of the employee using one or more artificial intelligence models. At step 910, the AI engine determines a remedial action to be rendered to the employee to improve the emotional state of the employee upon a predetermined criterion being met by the response of the employee. In an embodiment herein, the remedial action may include for example, a remedial content being displayed to the employee, for example the AI engine may gather videos or audios or text that could be motivational for the employee and can be personalized based on the employee assessment data and may be rendered to the employee via the user interface. In some other embodiment herein, the remedial action may include for example, an action to be taken by a manager, for example, a human resource manager may be intimated to speak to the particular employee to motivate the employee. In some other embodiments herein, some other remedial actions may be determined by the AI engine based on the needs of the employee. A representative hardware environment for practicing the embodiments herein is depicted in FIG. 10 with reference to FIGS. 1 through 9. This schematic drawing illustrates a hardware configuration of system 100 of FIG. 1, in accordance with the embodiments herein. The user device 202a-n includes at least one processing device 10. The special-purpose CPUs 10 are interconnected via system bus 12 to various devices such as a random-access memory (RAM) 14, read-only memory (ROM) 16, and an input/output (1/O) adapter 18. The I/O adapter 18 can connect to peripheral devices, such as disk units 11 and tape drives 13, or other program storage devices that are readable by the system. The user device 202a-n can read the inventive instructions on the program storage devices and follow these instructions to execute the methodology of the embodiments herein. The user device 202a-n further includes a user interface adapter 19 that connects a keyboard 15, mouse 17, speaker 24, microphone 22, and/or other user interface devices such as a touch screen device (not shown) to the bus 12 to gather user input. Additionally, a communication adapter 20 connects the bus 12 to a data processing network 25, and a display adapter 21 connects the bus 12 to a display device 23, which provides a graphical user interface (GUI) 29 of the output data in accordance with the embodiments herein, or which may be embodied as an output device such as a monitor, printer, or transmitter, for example. Further, a transceiver 26, a signal comparator 27, and a signal converter 28 may be connected with the bus 12 for processing, transmission, receipt, comparison, and conversion of electric or electronic signals. The user device 202a-n may also include an image capture unit 31, such as a camera for capturing image or video of the users. The embodiments herein can include both hardware and software elements. The embodiments herein that are implemented in software include but are not limited to, firmware, resident software, microcode, etc. Furthermore, the embodiments herein can take the form of a computer program product accessible from a computer-usable or computer-readable medium providing program code for use by or in connection with a computer or any instruction execution system. For the purposes of this description, a computer-usable or computer readable medium can be any apparatus that can comprise, store, communicate, propagate, or transport the program for use by or in connection with the instruction execution system, apparatus, or device.

The system, method, computer program product, and propagated signal described in this application may, of course, be embodied in hardware: e.g., within or coupled to a Central Processing Unit “CPU”), microprocessor, microcontroller, System on Chip “SOC”), or any other programmable device. Additionally, the system, method, computer program product, and propagated signal may be embodied in software (e.g., computer readable code, program code, instructions and/or data disposed in any form, such as source, object, or machine language) disposed, for example, in a computer usable (e.g., readable) medium configured to store the software. Such software enables the function, fabrication, modeling, simulation, description and/or testing of the apparatus and processes described herein.

Such software can be disposed in any known computer usable medium including semiconductor, magnetic disk, optical disc (e.g., CD-ROM, DVD-ROM, and the like) and as a computer data signal embodied in a computer usable (e.g., readable) transmission medium (e.g., carrier wave or any other medium including digital, optical, or analog-based medium). As such, the software can be transmitted over communication networks including the Internet and intranets. A system, method, computer program product, and propagated signal embodied in software may be included in a semiconductor intellectual property core (e.g., embodied in HDL) and transformed to hardware in the production of integrated circuits. Additionally, a system, method, computer program product, and propagated signal as described herein may be embodied as a combination of hardware and software.

A “computer-readable medium” for purposes of embodiments of the present invention may be any medium that can contain, store, communicate, propagate, or transport the program for use by or in connection with the instruction execution system, apparatus, system or device. The computer readable medium can be, by way of example only but not by limitation, an electronic, magnetic, optical, electromagnetic, infrared, or semiconductor system, apparatus, system, device, propagation medium, or computer memory”

A “processor” or “process” includes any human, hardware and/or software system, mechanism or component that processes data, signals or other information. A processor can include a system with a general-purpose central processing unit, multiple processing units, dedicated circuitry for achieving functionality, or other systems. Processing need not be limited to a geographic location or have temporal limitations. Portions of processing can be performed at different times and at different locations, by different (or the same) processing systems.

The various embodiments of the present technology provides an automated system that can assess mood, energy and emotion of the employees and also serve as an independent web-based app that also integrates with point-of-sale, scheduling and time tracking systems. The present technology captures a daily pulse of employee sentiment that provides a single source of truth, delivering actionable insights into employee state. Using the present technology, the managers can quickly identify potential problem areas and take targeted actions based on real-time data. Additionally, the dashboards provide data tools, training and prescriptive solutions driven by machine learning algorithms. The present technology enables the managers to set alerts that are triggered based on threshold scores of inputs by the employees.

The foregoing description of the specific embodiments herein will so fully reveal the general nature of the embodiments herein that others can, by applying current knowledge, readily modify and/or adapt for various applications such as specific embodiments herein without departing from the generic concept, and, therefore, such adaptations and modifications should and are intended to be comprehended within the meaning and range of equivalents of the disclosed embodiments herein. It is to be understood that the phraseology or terminology employed herein is for the purpose of description and not of limitation. Therefore, while the embodiments herein have been described in terms of preferred embodiments herein, those skilled in the art will recognize that the embodiments herein can be practiced with modifications. However, all such modifications are deemed to be within the scope of the claims. The scope of the embodiments herein will be ascertained by the claims to be submitted at the time of filing a complete specification.

What is claimed are:

1. A processor-implemented method for automated assessment of emotional state of an employee using artificial intelligence, the method comprising steps of:
receiving a login request by an employee on an automated assessment platform;
generating an actionable prompt via an artificial intelligence engine (AI engine) associated with a server based on one or more attributes associated with the employee and providing the actionable prompt to the employee via a user interface by the automated assessment platform upon login by the employee;
capturing employee assessment data, by the automated assessment platform, based on one or more actions of the employee in response to the actionable prompt and transmitting the employee assessment data to the AI engine;
analyzing the employee assessment data, by the AI engine, to assess the emotional state of the employee using one or more artificial intelligence models; and
determining a remedial action for the employee, by the AI engine, based on analysis, to facilitate maintaining of emotional state of the employee.

2. The processor-implemented method of claim 1, wherein the step of generating the actionable prompt comprises:
analyzing a previously gathered data associated with the employee and one or more attributes associated with the employee by the AI engine; and
generating the actionable prompt in real-time for each individual employee based on the analysis.

3. The processor-implemented method of claim 1, wherein the actionable prompt is generated based on geofencing.

4. The processor-implemented method of claim 1, further comprises:
progressively learning by the AI engine about the employee based on the employee assessment data collected over a period of time and predict one or more attributes associated with the employee based on the learning.

5. The processor-implemented method of claim 1, further comprises:
calculating an employee performance and/or sentiment score by the AI engine, based on one or more data points associated with the captured actions; and
determining the personalized remedial content suitable for the employee for improving the emotional state and/or performances of the employee.

6. The processor-implemented method of claim 1, further comprises:
identifying by the AI engine one or more patterns in the employee assessment data that is predictive of performance and/or sentiment of an employee using the employee assessment data, using one or more machine learning techniques.

7. The processor-implemented method of claim 1, further comprises:
generating one or more alert/notification signals by the automated assessment platform, upon the employee performance and/or sentiment score being below a predetermined threshold.

8. The processor-implemented method of claim 1, wherein capturing employee assessment data comprises:
capturing at least one of an identification data comprising at least one of: a facial recognition data, a voice recognition data, a fingerprint data, of the employee upon the employee logging into the automated assessment platform; and
transmitting the identification data to the AI engine.

9. The processor-implemented method of claim 8, further comprises:
determining by the AI engine an emotional state of the employee based on the identification data and triggering a notification based on the emotional state determined.

10. The processor-implemented method of claim 9, further comprises:
capturing a narrative response to one or more questions, from the employee upon the employee logging into the automated assessment platform;
transmitting the narrative to the AI engine; and
determining by the AI engine an emotional state of the employee based on the narrative data and triggering a notification based on the emotional state determined.

11. The processor-implemented method of claim 1, further comprises:
weighing, by the AI engine, one or more individual scores corresponding to at least one of: mood, physical energy, and/or emotions, of the employee, based on one or more statistical techniques and models.

12. The processor-implemented method of claim 1, wherein determining the remedial action comprises:
selecting a remedial content to be displayed to at least one of: the employee and the manager.

13. The processor-implemented method of claim 1, wherein determining the remedial action comprises:
determining an action to be taken by a manager; and
intimating the manager by the automated assessment platform to perform the action.

14. The processor-implemented method of claim 1, wherein the actionable prompt comprises audio and/or video content displayed to the employee, wherein a response from the employee in the form of at least one of: a) video, b) audio or c) facial expression of the employee in response to the audio and/or video displayed to them, is captured by the automated assessment platform for assessment.

15. The processor-implemented method of claim 1, wherein the actionable prompt is personalized in real-time by the AI engine based on one or more attributes associated with the employee.

16. The processor-implemented method of claim 1, wherein the AI engine is configured to:
analyze an aggregate employee performance or emotional state score, either alone, or together with certain previously aggregated employee performance or emotional state scores for at least one of: same or similar groups of employees; and
generate the actionable prompts in real-time for each individual employee based on the analysis.

17. The processor-implemented method of claim 1, wherein the AI engine is further configured to:
analyze an aggregate employee performance or emotional state score, either alone, or together with certain previously aggregated employee performance or emotional state scores for a group of employees; and
generate at least one of: a remedial content and one or more actions to be taken by a supervisor or management, for improving the emotional state of the group of employees.

18. A method of training an artificial intelligence engine for automated assessment of emotional state of an employee using artificial intelligence, the method comprising steps of:
providing an actionable prompt to an employee via a user interface by an automated assessment platform upon login by the employee on the automated assessment platform;
receiving one or more actions from the employee in response to the actionable prompt;
capturing employee assessment data based on one or more actions of the employee on the actionable prompt; and
progressively training one or more machine learning models associated with an artificial intelligence engine using the captured employee assessment data, for assessing the emotional state of the employee using the one or more artificial intelligence models;
wherein the one or more machine learning models are trained progressively based on employee assessment data captured in a plurality of instances over a predetermined period of time to identify one or more patterns that are predictive of performance of the employees.

19. A system for automated assessment of emotional state of an employee using artificial intelligence, the system comprising:
an automated assessment platform comprising a processor and a memory comprising one or more executable modules executable by the processor for enabling, automated assessment of emotional state of an employee using artificial intelligence, wherein the automated assessment platform is configured for:
a) receiving a login request by an employee on an automated assessment platform:
b) rendering an actionable prompt to the employee via a user interface; and
c) capturing employee assessment data, based on one or more actions of the employee on the actionable prompt and transmitting the employee assessment data to an artificial intelligence (AI) engine associated with a server;
the server communicatively coupled to the automated assessment platform and comprising the AI engine and a database, wherein the AI engine is configured to:
a) generate the actionable prompt in real-time based on one or more attributes of the employee, upon login by an employee on the automated assessment platform; and,
b) receive employee assessment data, based on one or more actions of the employee on the actionable prompt;
c) analyze the employee assessment data, to assess the emotional state of the employee using one or more artificial intelligence models; and
d) generate a remedial content to be rendered to the employee based on analysis, to facilitate maintaining of emotional state of the employee.

20. The system of claim 19, wherein the AI engine is further configured to:
analyze a previously gathered data associated with the employee and one or more attributes associated with the employee by the AI engine; and
generate the actionable prompt in real-time for each individual employee based on the analysis.

21. The system of claim 19, wherein the AI engine is further configured to:
progressively learn about the employee based on the employee performance data collected over a period of time and predict employee performance.

22. The system of claim 19, wherein the AI engine is further configured to:
calculate an employee performance score based on one or more data points associated with the captured actions; and
determine the personalized remedial content suitable for the employee for improving the emotional state of the employee.

23. The system of claim 19, wherein the AI engine is further configured to:
identify one or more patterns in the employee assessment data that is predictive of performance an employee using the employee assessment data, using one or more machine learning techniques.

24. The system of claim 19, wherein the automated assessment platform is further configured to:
generate one or more alert/notification signals by the automated assessment platform, upon the employee performance score being below a predetermined threshold.

25. The system of claim 19, wherein the automated assessment platform is further configured to:
capture a facial recognition data of the employee upon the employee logging into the automated assessment platform; and
transmit the facial recognition data to the AI engine.

26. The system of claim 19, wherein the AI engine is further configured to:
determine an emotional state of the employee based on the facial recognition data and triggering a notification based on the emotional state determined.

27. The system of claim 19, wherein the AI engine is further configured to:
weigh individual scores for mood, physical energy, and/or emotions based on one or more statistical techniques and models.

28. The system of claim 19, wherein the AI engine is further configured to:
select a remedial content to be displayed to at least one of: the employee and the manager.

29. The system of claim 19, wherein the AI engine is further configured to:
determine an action to be taken by a manager; and
intimate the manager via the automated assessment platform to perform the action.

30. The system of claim 19, wherein the employee assessment data comprises an aggregate employee data that is an aggregate for a shift, wherein the AI engine analyses the aggregate data to provide suggestion to the manager on how to improve the overall energy of the employees corresponding to the shift.

31. The system of claim 30, wherein the aggregate employee data is as an aggregate for at least one of: an entire department, location or an entire company.
